(19) United States
(12) Patent Application Publication
(10) Pub. No.: US 2009/0137538 A1
(43) Pub. Date: May 28, 2009

(54) METHOD OF TREATING ATROPHIC VAGINITIS

(76) Inventors: Bernadette Klamerus, Cambridge, MA (US); Janet A. Chollet, Pittsburgh, PA (US)

Correspondence Address: WILMERHALE/BOSTON, 60 STATE STREET, BOSTON, MA, 02109, US

(21) Appl. No.: 12/163,319

(22) Filed: Jun. 27, 2008

Related U.S. Application Data
(63) Continuation of application No. 11/625,675, filed on Jan. 22, 2007.
(60) Provisional application No. 60/760,440, filed on Jan. 20, 2006.

(51) Int. Cl. A61K 31/56

(52) U.S. Cl. 514/170; 514/182; 514/177

(57) ABSTRACT

This invention relates to a method and pharmaceutical composition useful in treating a condition responsive to hormone replacement therapy. Specifically, the invention is related to the long term treatment of symptoms associated with atrophic vaginitis. The composition contains effective amounts of an estrogen, a progesterone compound and a pharmaceutically accepted vehicle, carrier and/or diluent.

### CROSS-REFERENCE TO RELATED APPLICATIONS

This application is a continuation of and claims priority under 35 U.S.C. § 120 to U.S. application Ser. No. 11/625,675, filed on Jan. 22, 2007, which claims priority under 35 U.S.C. § 119 to U.S. Provisional Application Ser. No. 60/760,440, filed on Jan. 20, 2006, the disclosures of which are incorporated herein by reference in their entirety.

### FIELD OF THE INVENTION

The present invention relates to pharmaceutical compositions using a combination of an estrogen and progesterone as a vaginal therapy for the treatment of symptoms associated with atrophic vaginitis.

### BACKGROUND OF THE INVENTION

Atrophic vaginitis is a hormone-dependent disease involving the genital tract and lower urinary tract. Generally, atrophic vaginitis becomes evident during or after menopause, the symptoms increasing with age. Symptoms relating to urogenital aging are due to estrogen loss from follicular depletion in the menopausal ovary. This estrogen loss accounts for the majority of the anatomical, cytological, bacteriologic, and physiologic genital changes that occur in the vagina and lower urinary tract.

With estrogen loss, the vagina shortens, narrows, and the vaginal walls become thinner, less elastic and pale in color. Numerous symptoms accompany these changes. Collectively, the vaginal symptom complex is referred to as atrophic vaginitis. Unlike vasomotor symptoms, atrophy-related problems such as dyspareunia, burning and chronic vaginitis do not disappear with time. Irritation and burning are frequently a result of a chronic discharge caused by pH elevations and bacteriologic changes of the vaginal vault. Itching, which often interferes with a restful sleep, results from thinning and inflammation of the vulvovaginal epithelial layer. Vaginal pressure can be due to atrophy of the pelvic support ligaments due to a decrease in tissue collagen. Vaginal dryness occurs as the atrophic vagina produces less secretions. The vaginal surface thus becomes friable, with petechiae, ulcerations, and bleeding often occurring after minimal trauma.

It has been suggested that about 50% of otherwise healthy women over 60 years of age have symptoms related to vaginal atrophy (Iosif et al., Acta Obstetricia et Gynaecologica Scandinnavica 1984; 63: 257-60). Dennerstein and colleagues examined the prevalence of vaginal dryness among 438 women over a 7-year follow-up period and found that vaginal dryness begins to appear before perimenopause, increases during the early perimenopausal period, and significantly increases within 2 to 3 years after menopause (Dennerstein et al., Obstet Gynecol 2000; 96: 351-358). Overall, in about 45% of menopausal women, vaginal atrophy can clinically manifest as a syndrome of vaginal dryness, itching, irritation and dyspareunia (Bygdeman et al., Maturitas 1996; 23: 259-63). The vaginal symptoms range in severity from minor annoyance to debilitating. In the United States, 20 million women, who do not undergo estrogen hormone therapy, will have socially disabling symptoms related to urogenital atrophy (Samsioe, Am J Obstet Gynecol 1998; 178: S245-S249).

The epithelial changes in the bladder are similar to those occurring in the vagina and result in thin, pale, friable tissue. Specifically, the lower urinary symptoms include dysuria, frequency, urgency, and incontinence (Simunic, et al. Int J Gynaecol Obstet 2003; 83: 187-197). At least one symptom is reported by 40% of menopausal women (Barlow, et al. Maturitas 1997; 27: 239-247). Overactive bladder, which is a clinical syndrome defined as “urgency” or “frequency” with or without urge incontinence, usually with frequent nocturia (Abrams, et al. Neurourol Urodyn 2002; 21: 167-178).

Overactive bladder has been shown to have a negative impact on quality of life. Sexual dysfunction, which includes decrease sexual desire, frequency of sexual activity and sexual satisfaction is more common in women with overactive bladder than in those without (Yip, et al. Am J Obstet Gynecol. 2003; 188: 1244-1248). The nocturia that is often experienced with overactive bladder diminishes quality of sleep (Stewart, et al. World J Urol. 2003; 20: 327-336). Subsequently, the increased need to void at night has been shown to increase the risk for falling and a hip fracture in elderly osteoporotic women (Brown, et al. J Am Geriatr Soc. 2000; 48: 721-725). Overactive bladder also poses a heavy financial burden to the healthcare community as a whole. In the U.S., the overall costs associated with overactive bladder is greater than 9 billion dollars annually (Hu, et al. BJU Int. 2005; 96(suppl 1): 43-45).

The present treatment options for overactive bladder include observation/do nothing, pads/diapers, medical therapy, sacral stimulation and surgical reconstruction. The most common management of an overactive bladder consists of administering a smooth muscle relaxant, such as antimuscarinic agents, which acts directly on the smooth muscle. Existing treatments are known to have a number of side effects thus limiting its use due to discontinuation of the agent. The potential side effects of all antimuscarinic agents include inhibition of salivary secretions (dry mouth), gut motility (constipation), blockade of the sphincter muscles of the iris and the ciliary muscle of the lens (blurry vision), drowsiness, cognitive dysfunction, and inhibition of sweat gland activity. In general, antimuscarinic agents in patients with narrow angle glaucoma should be used with caution in patients with significant bladder outlet obstruction and gastric motility disorders. For a summary of data on adverse events, see Table 1.

TABLE 1
Adverse Events for Antimuscarinic Agents Compared With Placebo

| | | Blurred | | | Dry | | Urinary |
|---|---|---|---|---|---|---|---|
| Drug and Dose | Any AE* | Vision | Constipation | Dizziness | Mouth | Dyspepsia | Retention* |
| Tolterodine IR | X | X | X | X | 2.4 (15, | X | X |
| 2 mg | | | | | 4.0) | | |
| Tolterodine IR | X | X | X | X | 3.6 (2.9, | X | X |
| 4 mg | | | | | 4.4) | | |
| Tolterodine ER | X | X | X | X | 2.9 (2.3, | X | X |
| 4 mg | | | | | 3.7) | | |
| Oxybutynin 1R | | X | X | | X | | |
| 5-7.5 mg | | | | | | | |
| Oxybutynin IR | 1.4 (1.1, | 1.7 (1.1, | X | X | 3.3 (2.3, | 3.3 (1.5, | 5.6 (1.9, |
| 8.8-15 mg | 1.7) | 2.6) | | | 4.7) | 7.1) | 17.0) |
| Oxybutynin | X | X | X | X | X | | |
| TDS 3.9 mg | | | | | | | |
| Darifenacin | 1.2 (1.1, | | 2.2 (1.1, | | 2.2 (1.3, | X | |
| 7.5 mg | 1.5) | | 4.1) | | 3.9) | | |
| Darifenacin | 1.4 (1.1, | | 2.4 (1.5, | | 2.9 (1.7, | 3 2 (1.0, | |
| 15 mg | 1.6) | | 3.9) | | 1.8) | 10.2) | |
| Solifenacin | X | X | 2.9 (1.5, | | 3.0 (1.9, | X | |
| 5 mg | | | 5.7) | | 4.6) | | |
| Solifenacin | X | 2.4 (1.3, | 4.4 (2.4, | | 5.8 (3.6, | X | |
| 10 mg | | 4.2) | 8.3) | | 9.3) | | |
| Trospium | 1.5 (1.0, | | 2.1 (1.4, | X | 3.2 (2.4, | | |
| 40 mg | 2.1) | | 3.2) | | 4.2) | | |

All cells with data report statistically significant relative risk ratios favoring placebo.
Blank cells = Data were not suitable for meta-analysis
X = No statistically significant difference for the intervention compared with placebo
*Trial definition
From: Chapple C. Eur. Urol. 2005, 48: 5-26.

It has been shown that the use of the hormone estriol dramatically reduces urinary tract infections and urge incontinence thus markedly improving the quality of life in elderly patients (Molander et al., Maturitas 1990; 12: 113-120; Samsioe et al., Maturitas 1985; 7: 335-342; and Luisi et al., Maturitas 1980; 2: 311-9). Estriol therapy restored premenopausal vaginal flora in women with recurrent urinary tract infections, reducing the requirement for antibiotics by up to 16 times compared to those unsupplemented (Brandberg et al., Acta Obstet Gynecol Scand 1984; 140:33).

In addition to urinary tract infections, estrogen deficiency seen during menopause is thought to affect urinary control by lowering the urethral closure pressure and increasing the awareness of bladder fullness thereby causing urge incontinence or an overactive bladder (Cardoza, et al. Gynecol Endocrinol 1995; 9: 75-84). Menopausal women benefit from estrogen therapy because it improves the vasculature of the bladder neck and the mucosa of the urethra. Previous studies have shown the presence of estrogen receptors in the trigone and proximal urethra (Cardoza, et al. Gynecol Endocrinol 1995; 9: 75-84; Versi E. Clin Obstet Gynecol 1990; 33: 392-7). These findings provide evidence of a direct action of estrogen on the lower urinary tract that was subsequently considered important in the pathogenesis and management of urinary control in menopausal women.

Unfortunately, only a small percentage, about ten percent, of those who would benefit from estrogen therapy actually receive it for many reasons. For example, women are embarrassed to volunteer to their doctor or health care professional that they have significant vaginal symptoms, such as painful intercourse (Notelovitz, Intl J Gyn Obstet 1997; 59:S35-9). Women also have been very reluctant to take hormone replacement therapy because of the results of a recent clinical trial. The harmful impact of hormone replacement therapy became evident to the health care community at large and to the general public based upon the results of the PEPI study (Writing Group for the PEPI Trial, Effects of hormone replacement therapy on endometrial histology in postmenopausal women. The Postmenopausal Estrogen/Progestin Interventions (“PEPI”) Trial, JAMA 1996; 275: 370-5). Patients in the PEPI trial were randomized in a double-blinded, placebo-controlled fashion with three years of follow-up. The trial assessed the effects of oral hormone replacement therapy on a number of parameters, including its activity on the endometrium. The trial involved 596 women who were specifically randomly assigned to either a placebo, estrogen only, or one of three estrogen/progesterone regimen arms. Histological data revealed that ten (10%) percent of women taking unopposed estrogen therapy (equivalent to 0.625 mg conjugated equine estrogen (“CEE”)) would develop complex or atypical hyperplasia within one year. Combining CEE with cyclic or continuous progesterone protected the endometrium from hyperplastic changes associated with estrogen-only therapy alone. This study represented the first unequivocal demonstration of the importance of developing and optimizing combination therapy utilizing dosing regimens that select for both safety and efficacy.

The concept of administering a vaginal estrogen with progesterone to prevent endometrial hyperplasia is less accepted by the medical community, despite a significant systemic rise in serum estrogen levels (Martin et al., JAMA 1979; 242: 2699-700; Mandel et al., J Clin Endocrinol Metab 1983; 57: 133-9). Tourgeman and colleagues reported ten-fold higher serum estradiol serum levels after vaginal versus oral administration of estradiol, while endometrial concentrations were seventy-fold higher given the same exact dose (Tourgeman et al., Am J Obstet Gynecol 1999; 180:1480-1483).

The observation of a significant rise in progesterone receptors after the administration of vaginally delivered estriol and estradiol therapy further supports the observation of its estrogenic effect on the endometrium. The increase number of progesterone receptors is recognized as a biochemical signal for prolonged estrogenic influence on estrogen sensitive tissue (Leavitt et al., Ann. N.Y Acad. Sci., 286, 210-25; Horwitz et al., J Biol. Chem. 1978, 253:2223-8; Clark, J. H. and Peck, E. J., In: Female Steroids, Receptors and Function 1979, (Gross et al. (eds), Berlin: Springer Verlag) p. 103-14). An estrogenic effect on the endometrium is seen with the vaginal ring birth control method, which is used in fertile women (Timmer et al., Clin Pharm 2000; 39:233-242). The hormones are rapidly and continuously absorbed when the ring is placed into the vagina. The bioavailability of ethinylestradiol in the vaginal ring after vaginal administration is approximately 55.6%, which is comparable to that with oral administration of ethinylestradiol. Thus, it is evident that vaginally delivered birth control has systemic absorption as does vaginally delivered hormone replacement therapy.

It is well documented that vaginal estrogen therapy has been associated with endometrial proliferation and hyperplasia (Luisi et al., Maturitas 1980; 2: 311-9; Widholm et al., Ann Chir Gynaecol Fenn 1974; 63: 186-90). As a result, the American College of Obstetricians and Gynecologists (ACOG) has recommended concomitant progestin therapy for women receiving a vaginal estrogen (ACOG, Hormone replacement therapy 1992, ACOG technical bulletin No. 93., Washington, D.C.). Recently, the ACOG suggested using a lower dose of estrogen (0.3 mg) of conjugated equine estrogen (Premarin®), which is also referred to as a low potency formulation (ACOG, Genitourinary Tract Changes 2004, Vol. 104, No. 4 Supplement, Washington, D.C.). The goal was to deliver estrogen with the hope that this regimen would be associated with a lower incidence of endometrial pathology, but unfortunately this has failed to achieve this clinical benefit.

The data using a low dose 0.3 mg of conjugated equine estrogen (CEE) given vaginally suggests that women who use even a low dose of unopposed vaginal estrogen may be at an increased risk of endometrial carcinoma with long-term use (Handa et al., Obstet Gynecol 1994; 84: 215-8). The data using oral CEE demonstrated a dose-related increase in incidence rates of endometrial hyperplasia from 3.17% (oral conjugated estrogens 0.3 mg/d) to 14.9% (oral conjugated estrogens 0.45 mg/d) to 27.27% (oral conjugated estrogens 0.625 mg/d) within 2 years. (Utian et al., Fertil Steril 2001; 75: 1065-79). Due to reports on the effect of Premarin® on the endometrium, the product information in the prescribing guide continues to recommend that practitioners give progesterone in conjunction with the estrogen in order to shed any uterine tissue, which may have built up as a result of unopposed estrogen therapy.

It is also evident that there is no lower incidence of endometrial pathology with the use of other low potency unopposed estrogen formulations as recommended by ACOG. This is supported by the histological observation of uteri from hysterectomized women. Three-week vaginal application of either estriol (0.5 mg estriol) or estradiol (0.05 mg estradiol) contributed to overstimulation of the endometrium with low potency formulations (Van Haaften et al., Gynecol. Endocrinol 1997; 11: 175-185). Data indicating an estrogenic effect of vaginal application of estriol (0.5 mg for 16 days) on the uterus seen by scanning electron microscopy further supports the argument that vaginal unopposed low potency formulations may have an adverse endometrial effect (Englund et al., Acta Obstet. Gynecol. Scand. 1982, 106 (Suppl.): 23-6). A study of women with uterine prolapse awaiting hysterectomy had endometrial atrophy as determined by histological exam were treated with oral estriol 2 mg per day for an average three weeks prior to hysterectomy. On histological exam of the uteri post-hysterectomy, there were hyperplastic changes in 70.8% of the women (Montoneri et al., Clin Exp Obst Gyn 1987, 14:178-181). Evidence continues to show an increased relative risk of endometrial cancer in postmenopausal women who use oral estriol. The relative risk increased with duration of use, and there was a greater increase in relative risk for endometrial atypical hyperplasia with an odds ratio of 1.0 for never use and those exposed to hormones for less than 5 years having an odds ratio of 2.2. There was an odds ratio of 8.3 when treatment exceeded 5 years. In the same study with vaginally administered low potency formulation, there was an odds ratio of 1.0 for never use compared to an odds ratio of 2.3 for atypical hyperplasia with at least five years of use (Weiderpass et al., Lancet 1999; 353: 1824-8). More evidence has shown an increased risk of endometrial hyperplasia after vaginal use of low potency formulations (Barensten et al., Eur J Obst & Gyn and Reprod Bio 1997; 71: 73-80; Dugal et al., Acta Obststricia et Gynecologica Scandinavica 2000; 79: 293-7; Kelsey et al., Am J Epidemiol 1982; 116: 333-42). Hence, due to reports on the effect of low potency formulation on the endometrium, it is recommended for practitioners to prescribe progesterone in conjunction with estrogen therapy in order to shed any uterine tissue, which may have built up as a result of the low potency formulation (Head, Alt Med Rev 1998; 3(2): 101-113).

Overall, it is desirable to use estrogen to treat a variety of endocrine disorders. However, it is well known that these compounds are not suitable for oral administration due to first pass effect and metabolism. These hormones are carried by the portal system to the liver leading to metabolism and rapid elimination of the estrogens. Because of liver metabolism into inactive ingredients, effective oral administration has required excessively high dosage levels. In the past, different routes of administration have been developed in an attempt to improve upon both safety and efficacy. The development of numerous steroidal derivatives of estrogen administered parenterally, by injection, transvaginal (creams, tablets, and silastic rings), transdermal (“patch”), and subcutaneous pellets, intranasal, and percutaneous (gel) have led to products that circumvent first pass metabolism. This has led to the ability to deliver clinically effective steroids.

In the past, customary usage of estrogen and progesterone for treating menopause has involved sequential administration. This method of administration has been poorly tolerated because it often results in withdrawal bleeding experienced by the patient as a menstrual period and therefore, not well-tolerated, often leading to discontinuation of therapy. Unfortunately, patients are forced to suffer because of the unacceptability of treatment. Whereas, a continuous regimen of combination hormone therapy has been used in an attempt to reduce the incidence of withdrawal bleeding and achieve amenorrhea. Bleeding is a major concern of older postmenopausal women. A continuous regimen in this group of women is least likely to have bleeding hence maintaining the benefits of hormone replacement therapy.

With the advancing age of the American population as amplified by the entry of the baby-boom generation into their climacteric years, the need for safe and effective hormone replacement therapy is imperative and important to addressing the health and well-being of aging women. The CDC reported in 2004, a jump in the number of older Americans with AIDS (AIDS Policy LAW 2004 Mar. 26; 19 (6): 4). Since, 1991, AIDS cases among those 50 and older have jumped by more than 22 percent, according to a report by the Centers for Disease Control and Prevention. This jump might be explained secondary to more sexually active women entering the climacteric years with a diagnosis of atrophic vaginitis. Recent data in women have strongly suggested the relevance of an atrophic vagina and increased rate of HIV infection. Smith and colleagues demonstrated that estriol-treated animals were strongly protected against SIV vaginal transmission (8.3% infection rate) compared with animals treated with base cream alone (75% infection rate) (Smith et al., AIDS 2004; 18: 1637-1643). In human data, women with suppressed estrogen levels had a two- to three-fold increased rate of HIV infection (Martin et al., J Infect Dis 1998, 178: 1053-1059). The human data and the data derived in the macaque model support the hypothesis that the vaginal epithelium is a natural an important barrier against HIV infection in women and that hormonal alterations of this barrier can enhance (estrogen) its protective effects. The combined record of estriol safety in women, and data on risk factors of HIV vaginal transmission support the use of vaginal estriol in women who have low levels of estrogen, to reduce their risk of heterosexual transmission.

There is a clear need in the art to provide an effective and safe vaginally administered hormone therapy to treat menopausal symptoms, including atrophic vaginitis, and which avoids the adverse effects associated with the long-term systemic absorption of a local unopposed estrogen therapy and that lessens the adverse events that accompany antimuscarinic agents. The preferred route of administration in treating symptoms associated with atrophic vaginitis would be intravaginal as it is the target tissue and that there is a direct local effect on lower urinary tract. However, the effect of the combination of progesterone and estrogen given vaginally as a hormone replacement therapy in a single dosage unit is unknown; an intravaginal active formulation containing estrogen and progesterone in a single dosage unit has never been developed. The present invention, based on new clinical observations, addresses this need by providing a novel pharmaceutical composition that combines estrogen and progesterone in a single unit dosage form. Moreover, the invention describes both a safe and clinically effective formulation necessary to treat atrophic vaginitis symptoms resulting from surgical menopause, iatrogenic menopause, natural menopause, and conditions leading to Amenorrhea (uterus present) manifesting as menopause.

### SUMMARY OF THE INVENTION

The invention relates to a pharmaceutical composition that is effective in the treatment of urogenital symptoms associated with atrophic vaginitis.

The pharmaceutical composition contains effective amounts of an estrogen compound, preferably micronized estriol, and a progesterone compound, preferably micronized progesterone. The effective amount of progesterone is effective to reduce the concomitant liability of adverse uterine effects associated with long-term unopposed estrogen administration. The composition may also contain pharmaceutically acceptable carriers, vehicles and/or diluents.

The invention also relates to a method of treating urogenital symptoms associated with atrophic vaginitis. The method comprises the administration of a pharmaceutical composition containing effective amounts of an estrogen compound, a progesterone compound, and pharmaceutically acceptable carriers, vehicles and/or diluents. The method of treating atrophic vaginitis substantially reduces the concomitant liability of adverse uterine effects associated with unopposed estrogen administration.

In a specific embodiment, the administration of the composition is continued for at least 3 months, at least 6 months, preferably at least 12 months, more preferably for at least 18 months, and most preferred for greater than 24 months.

In the specific embodiment, the composition is administered as a vaginal suppository. In another embodiment, the composition is administered as a vaginal cream.

These and other aspects of the invention are discussed more in the detailed description and examples.

### DETAILED DESCRIPTION

The present invention advantageously provides for a method and a pharmaceutical composition in the treatment of symptoms associated with hormone deficient disorders responsive to estrogen, such as atrophic vaginitis. The present invention provides a long-term treatment regimen, e.g., greater than three months of continuous treatment, up to greater than 24 months of continuous treatment, while minimizing and/or preventing health risks associated with hormone replacement therapies. The invention is based, in part, on the remarkable efficacy and safety of estriol, with micronized progesterone, in treating atrophic vaginitis.

The terms used in this specification generally have their ordinary meanings in the art, within the context of this invention and in the specific context where each term is used. Certain terms are defined below to provide additional guidance in describing the compositions and methods of the invention and how to make and use them.

### DEFINITIONS

The term “about” or “approximately” means within an acceptable error range for the particular value as determined by one of ordinary skill in the art, which will depend in part on how the value is measured or determined, i.e., the limitations of the measurement system. For example, “about” can mean within 3 or more than 3 standard deviations, per the practice in the art. Alternatively, “about” can mean a range of up to 20%, preferably up to 10%, more preferably up to 5%, and more preferably still up to 1% of a given value. Alternatively, particularly with respect to biological systems or processes, the term can mean within an order of magnitude, preferably within 5-fold, and more preferably within 2-fold, of a value.

The phrase “pharmaceutically acceptable” refers to molecular entities and compositions that are “generally regarded as safe” (GRAS), e.g., that are physiologically tolerable and do not typically produce an allergic or similar untoward reaction, such as gastric upset, dizziness and the like, when administered to an animal. Preferably, as used herein, the term “pharmaceutically acceptable” means approved by a regulatory agency of the Federal or a state government or listed in the U.S. Pharmacopeia or other generally recognized pharmacopeia for use in animals.

The term “carrier” refers to a diluent, adjuvant, excipient, or vehicle with which the compound is administered. Such pharmaceutical carriers can be sterile liquids, due to its high insolubility in water, oils, including those of petroleum, animal, vegetable or synthetic origin, such as peanut oil, soybean oil, mineral oil, sesame oil and the like. Carriers such as micelles or dextrans can be used to deliver the agent in an aqueous solution or suspension. Suitable pharmaceutical carriers are described in “Remington's Pharmaceutical Sciences” by E. W. Martin.

The term “amount” as used herein refers to quantity or to concentration as appropriate to the context. In the present invention, the effective amount of an estrogen compound refers to an amount sufficient to treat symptoms associated with atrophic vaginitis. The effective amount of a progesterone compound refers to an amount sufficient to counter the unwanted proliferative effects of the estrogen compound. The effective amount of a drug that constitutes a therapeutically effective amount varies according to factors such as the potency of the particular drug, the route of administration of the formulation, and the mechanical system used to administer the formulation. A therapeutically effective amount of a particular drug can be selected by those of ordinary skill in the art with due consideration of such factors.

As used herein, the term “urogenital” refers to the genital tract and the lower urinary tract, which are all part of the atrophic vaginitis syndrome.

### Pharmaceutical Formulation

### Estrogen Compounds

An “estrogen” or “estrogen compound” is defined herein as any of the structures described in the 11th edition of “Steroids” from Steraloids Inc., Wilton N.H., here incorporated by reference. Included in this definition are non-steroidal estrogens described in the aforementioned reference. Other estrogen compounds included in this definition are estrogen derivatives, estrogen metabolites, estrogen precursors, and selective estrogen receptor modulators (SERMs). Also included are mixtures of more than one estrogen or estrogen compound. Examples of such mixtures are provided in Table II of U.S. Pat. No. 5,554,601 (see column 6). Examples of estrogens having utility either alone or in combination with other agents are provided, e.g., in U.S. Pat. No. 5,554,601. β-estrogen is the β-isomer of estrogen compounds. α-estrogen is the α-isomer of estrogen components. The term “estradiol” is either α- or β-estradiol unless specifically identified. The term “E2” is synonymous with β-estradiol, 17β-estradiol, and β-E2. αE2 and α-estradiol is the αisomer of βE2 estradiol.

In a specific embodiment, the estrogen compound is estriol, preferably micronized estriol. Estriol is a naturally occurring steroidal sex hormone. It is an endogenous estrogen, formed primarily via peripheral metabolism of ovarian estrogens. Secreted ovarian estradiol is oxidized reversibly to estrone, both of which can be irreversibly converted to estriol. Most of estriol comes from estrone, though data has reported direct conversion of androstenedione to estriol without passing through the blood pool of estrone. Similar to other estrogens, estriol binds to intranuclear receptors after diffusing across the cell/nuclear membranes, with subsequent activation of selective messenger RNA synthesis; proteins/enzymes produced via the latter effect regulated specific cellular hormonal activity. Though differently to other estrogens, estriol does not bind to sex hormone-binding globulin (unlike estradiol and estrone), and thus has a short elimination half-life. Also, because most estradiol is bound to sex-hormone binding globulin (SHBG), only a portion of the circulating estradiol is available for entry into cells. On the other hand, estriol has a much lower affinity for binding to SHBG; therefore, a greater percent is available for biological activity.

Estriol is chemically described as 16-alpha, 17-beta, estra 1,3,5 (10) triene 3, 16, 17-triol. It has an empirical formula of C₁₈H₂₄O₃ and a molecular weight of 288.38. The structural formula is:

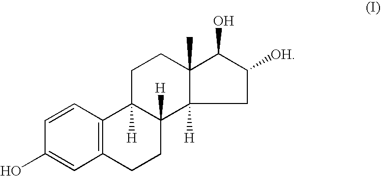

Estrogenic potency appears to be tissue specific. The downstream effect of activation of estrogen receptors is ligand dependent (McKenna et al., Endocr Rev 1999; 20:321-44; Kuiper et al., Endocrinology 1997; 138:863-70). In addition, the resulting ligand/receptor complex is not recognized in the same fashion by all cells, owing in part to the pattern of active genes and to steroid receptor co-regulators, which modulate the estrogen receptor (ER) of gene expression.

These finding explain how different ER ligands (estriol, tamoxifen and estradiol) manifest different responses in the same cell types and how the same ligand causes different responses in different cell types. For instance, the data demonstrates that tamoxifen (an estrogenic compound that competes with natural estrogen at receptor sites) protects against breast cancer but can cause uterine cancer. The data demonstrates that approximately 15 times more conjugated estrogens than estriol was needed to induce the same degree of vaginal maturation and cornification, which caused endometrial hyperplasia (Hustin et al., Acta Cytologica 1977; 21: 225-228). In the same study, estriol was less potent than conjugated estrogens in causing uterine growth (Phillips et al., Maturitas 1984; 5: 147-52).

Estriol is a more potent estrogen in the objective improvement of symptoms related to vaginal atrophy for it is highly efficacious in lowering the vaginal pH. It is well known that estrogen replacement therapy induces the normalization of the vaginal epithelium and therefore helps to restore the normal microflora and the physiological pH in the vagina, resulting in an increase in the resistance of the vaginal epithelial cells to infection. The decrease in circulating estrogen that occurs with menopause leads to a reduction in the glycogen content of the vaginal epithelial cells, which in turn inhibits the production of lactic acid by lactobacilli. Hence, vaginal pH is a useful indicator for the assessment of the vaginal epithelium and monitoring the effects of estrogen treatment in vaginal atrophy. With menopause, vaginal pH increases from the normal 3.5-4.0 (which favors lactobacilli) to 6.0-8.0 (which favors pathogenic organisms). Vaginal pH only decreased to 5.2 in the 0.3 mg conjugated estrogen group after 16 weeks of therapy (Marx et al., Maturitas 2004; 47: 47-54). Vaginal pH decreased to 4.8 in menopausal women treated with an estradiol-releasing ring for 24 weeks (Lose et al., BJOG 2000 August; 107(8): 1029-34), whereas, vaginal pH decreased markedly to 4.12 in menopausal women treated with estriol ovules at 1 mg for 24 weeks. (Dessole et al., Menopause 2004; 11: 49-56).

The ability of estriol to markedly lower the pH makes it an ideal agent in reducing the incidence of recurrent urinary tract infections in menopausal women. Urinary tract infections are very common in postmenopausal women, with 15% of women over 60 years old having frequent recurrent episodes. Local estrogen replacement therapy by means of intravaginally restores the atrophic vaginal, urethral and trigonal mucosae, stimulates the proliferation of lactobacilli and reduces pH, and as a consequence of these results, reduces colonization with Enterobacteriaceae and prevents bacteriuria. A significant decrease in vaginal pH and decrease in the rate of vaginal colonization with Enterobacteriaceae was observed with estriol therapy; lactobacilli (absent prior to therapy) reappeared after one month in 61% of patients given estriol but in no patients receiving placebo (Raz et al., N Engl J Med 1993; 329: 753-6). In addition, vaginal estriol therapy has been shown to be efficacious in alleviating urinary urgency (56%), urge incontinence (58%) and nocturia (54%) (Lose et al., BJOG 2000; 107(8):1029-34).

In the present invention, the amount of micronized estriol present in the composition depends on the strength of the final composition. In one embodiment, the micronized estriol is present in amounts ranging from about 0.01 mg to about 10 mg per dose, preferably from about 0.25 mg to about 1 mg per dose. The micronized estriol is preferably accompanied by a progesterone compound to reduce the concomitant liability of adverse uterine effects associated with long-term unopposed estrogen administration, particularly during menopause.

### Progesterone

Progesterone is a naturally occurring steroidal sex hormone and is defined as a compound that acts on the uterus to induce endometrial changes characteristic of pregnancy and that maintains pregnancy in animals. The progesterone receptor is under the dual control of estrogen and progesterone, which act sequentially to regulate cellular concentrations of progesterone receptor. The endometrial progesterone receptor is increased by estrogen via an estrogen-mediated increase in progesterone receptor messenger RNA levels and increased protein synthesis. It is down regulated by its own ligand, progestogen, at the transcriptional and posttranscriptional levels. In the human uterus, high concentrations of progesterone result in an inhibition of estrogen actions. The reduction in estrogen receptor synthesis is due to progestogen-mediated decrease in levels of estrogen receptor messenger RNA. Overall, by reducing the proliferative actions of estrogen, progesterone allows for differentiation to occur. Also, progestogens effectively lower estrogenic actions by down regulating estrogen receptors. It is thus the biochemical machinery, induced by estrogen, and the mitotic activity that have to be inhibited to prevent endometrial hyperplasia.

Progesterone has a chemical formula pregn-4-ene-3, 20-dione. It has a molecular weight of 314.47 and an empirical formula C₁₂H₃₀O₂. The structural formula is:

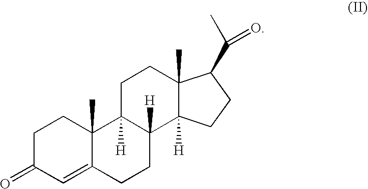

Progesterone compounds, which can be used in the present invention, include but are not limited to, progesterone (micronized progesterone) and progestin (synthetic progesterone).

Studies have shown that micronized progesterone (progesterone) is safer than synthetic progesterone (progestin) such as Medroxyprogesterone Acetate (MPA). Table 2 compares Medroxyprogesterone (MPA) versus Micronized Progesterone (MP), demonstrating the relative safety of MP over MPA.

TABLE 2

| | |
|---|---|
| Lipid Profile | MPA: adversely effects lipid profile and negates the beneficial |
| | effects of estrogen. |
| | MP: does not negate the beneficial effects of estrogen and modestly |
| | improves cholesterol levels. |
| Liver function | MPA: contraindicated in patients with liver dysfunction. |
| | MP: does not effect liver enzymes or cause liver related side |
| | effects. |
| Cardiovascular | MPA: may cause fluid retention and edema, increases incidence of |
| Events | CHD, stroke and VTE, and diminishes the cardio-protective effects |
| | of estrogens. |
| | MP: has antihypertensive action and can be safely used to treat |
| | preeclampsia. And with estrogen, prevents coronary vasospasms |
| | (in rhesus monkeys) and enhances the beneficial effects of estrogen |
| | on exercised-induced myocardial ischemia in menopausal women. |
| Glucose/Insulin | MPA: has been found to cause deterioration of glucose tolerance or |
| | hyperinsulemia or both. |
| | MP: augments the pancreatic response to glucose and increases the |
| | release of insulin. |
| Sleep and Mood | MPA: can cause insomnia, mental depression, and anxiety. |
| | MP: improves the quality of sleep and has sedative properties. |
| Quality of | When compared with MPA-containing regimen, women using MP- |
| life/menopausal | containing HRT experienced significant improvement in symptoms |
| symptoms | and 80% |

(The Writing Group for the PEPI Trial, JAMA, January 1995; 273:199-208; Physicians Desk Reference, 44^{th} edition, 1990; Bolaji, EUROBS (1993), 48:61-68; Darj, Gynecol. Endocrinol. (1993), 7:111-114; Rylance, Br Med J (Clin Res Ed) 1985, 290(6461):13-4; Sammour, Act Obstet Gynec Scand. 1975; 54:195-202; Sammour, Clin Exp Hyper-Hyper in Preg. 1982; B1: 455-78; Minshall et al., J of Clin Endocrin and Metabolism 1998, 83(2):649-59; Minshall et al., FASEB J 1998; 12(13):1419-1429; Rosano et al., J Am Coll Cardiol 2000: 36(7) p. 2154-9; Estrogen and Progestogens in Clinical Practice; Harcourt Brace & Co, 1998 ISBN 0443047065; Montplaisir, Menopause 2001; 8: 10-16; Arafat, Am J Obstet Gynecol 1998; 159: 1203-09; Fitzpatrick, J Women's Health & Gender-Based Medicine 2000; 9: 381-387).

In the present invention, micronized progesterone is the preferred progesterone compound. The amount of progesterone present in the composition may depend on the strength of the final composition. In one embodiment, the progesterone compound is present in amounts ranging from about 5 mg to about 500 mg per dose, preferably the range is from 25 mg to about 50 mg per dose, more preferably about 25 mg to about 30 mg per dose which is sufficient to oppose or inhibit the proliferative activity of the estrogen compound.

The aim of progesterone therapy is to prevent or limit endometrial hyperplasia associated with estrogen use. In order to do this, it is not necessary to induce a full secretory endometrium because a full secretory endometrium may produce an untoward side effect such as withdrawal bleeding. The lower doses of progesterone, which by design initially leave the endometrium partially secretory, may result in irregular bleeding or very light bleeding. However, the expected and desired result is amenorrhea, which will occur with time. Low doses of various progesterones, administered sequentially, such as an oral dose of 100 mg micronized progesterone are sufficient to inhibit endometrial estrogen receptor levels and mitotic activity (King et al., Fertil Steril 1986; 46: 1062-1066). Data has compared the bioavailability of orally and vaginally administered progesterone and the results showed that peak plasma progesterone concentrations for the two formulations were not significantly different making the two formulations having similar bioavailability (Norman et al., Fertil Steril 1991; 56: 1034-1039). Further, the use of transdermal progesterone (15 mg and 40 mg micronized progesterone) cream given twice daily had an equivalent antiproliferative effect on estrogen-stimulated postmenopausal endometrium (Leonetti et al., Fertil Steril 2003; 79: 221-22). Dosages of 100 mg micronized progesterone transvaginally more often induced (p<0.005 at six months and p<0.001 after 1 year) a functional like secretive endometrium causing a cyclic monthly cycle resulting in shedding of the endometrium (Ferrero et al., Minerva Ginecol 2002; 54: 519-30). Overall, the relative potency of an oral dose of 200 mg micronized progesterone is equivalent to that of a vaginal dose of 90 mg micronized progesterone. Given that an oral dose of 100 mg of micronized progesterone provides sufficient endometrial protection, an approximate dose of 45 mg of vaginal micronized progesterone should provide sufficient endometrial protection. Further, the serum concentration of 25 mg and 50 mg micronized progesterone administered as vaginal suppositories, were similar between both groups (7.27 ng/ml and 8.84 ng/ml respectively) (Von Eye Corleta et al., Gynecol Obstet Invest 2004; 58 (2): 105-8).

### Additional Constituents

The estrogen and progesterone compounds of the present invention may be formulated into a pharmaceutical composition with additional constituents for vaginal administration by way of suppositories, creams, foams, gels (including, but not limited to aqueous solutions and suspensions), ointments, tablets, ovules, pessaries and rings, and other known pharmaceutically acceptable carriers known in the art.

In one embodiment of the invention, the estrogen and progesterone are formulated with a fatty base. The base may be selected from, but is not limited to, JAB base, JC base, polyethylene glycol base, emollient cream, vanishing cream light, vanpen base, cosmetic HRT base, or mixtures thereof. When the mode of administration is through a vaginal suppository, preferably, the base is JAB base. JAB base is a combined formulation containing Base K, Base C and Base M or otherwise referred to as Bases B, J, and F, respectively. Base K is composed of PEG-8 distearate. Base C is composed of hydrogenated vegetable oil. Base M is composed of Vitamin E Acetate. The range for the JAB or BJF base in a suppository is from about 1.0 gm to about 1.40 gm, preferably about 1.28 gm. The weight of the active and inactive ingredients is about 300 mg or less.

When the compounds are formulated into a vaginal cream, the preferred base is JC base. The JC base is composed of an emollient or vanishing cream, including for example, PCCA Versabase and Base M.

Thus, the pharmaceutical composition may include one or more additives, depending on the pharmaceutically acceptable carrier, a preservative, a dye, a binder, a suspending agent, a dispersing agent, a colorant, a disintegrant, an excipient, a diluent, a lubricant, a plasticizer, an oil or any combination of any of the foregoing. Suitable pharmaceutically acceptable additives include, but are not limited to, ethanol; water; glycerol; aloe vera gel; allantoin; glycerin; vitamin A and E oils; mineral oil; PPG2 myristyl propionate; vegetable oils and solketal.

Suitable binders include, but are not limited to, starch; gelatin; natural sugars, such as glucose, sucrose and lactose; corn sweeteners; natural and synthetic gums, such as acacia, tragacanth, vegetable gum, and sodium alginate; carboxymethylcellulose; polyethylene glycol; waxes; and the like.

Suitable disintegrators include, but are not limited to, starch such as corn starch, methyl cellulose, agar, bentonite, xanthan gum and the like.

Suitable lubricants include, but are not limited to, sodium oleate, sodium stearate, magnesium stearate, sodium acetate, and the like.

The composition may also include suitable preservatives, e.g., sodium benzoate, and other additives the may render the composition more suitable for application, e.g., sodium chloride, which affects the osmolarity of the preparation.

Suitable dispersing and suspending agents include, but are not limited to, synthetic and natural gums, such as bentoite, vegetable gum, tragacanth, acacia, alginate, dextran, sodium carboxymethylcellulose, methylcellulose, polyvinyl-pyrrolidone and gelatin.

A suitable pharmaceutical diluent is, but is not limited to, water.

Examples of additional additives include, but are not limited to, sorbitol; talc; stearic acid; and dicalcium phosphate.

### Modes of Administration

Many methods may be used for vaginal administration of the formulation of the invention. These include vaginal administration of creams, suppositories, foams, gels (including, but not limited to aqueous solutions and suspensions), ointments, tablets, ovules, pessaries and rings. In certain embodiment of the invention, the estrogen and progesterone compounds may be formulated together or separately.

The effective dose may vary, depending upon factors such as the condition of the patient, the severity of the symptoms of the disease and the manner in which the pharmaceutical composition is administered. The compositions are formulated, preferably as per unit dose, or labeled for dispensing an amount, such that each dosage contains from about 0.01 mg to about 10 mg unit dose estrogen, and from about 5 mg to about 500 mg progesterone unit dose.

The pharmaceutical composition may be in a “unit dosage form”, which refers to physically discrete units suitable as unitary dosages for human subjects and other mammals, each unit containing a predetermined quantity of active material calculated to produce the desired therapeutic effect, in association with one or more of the above-described suitable pharmaceutical diluents, excipients or carriers.

### Methods of Treatment

The pharmaceutical compositions of the present invention may be administered to an animal, preferably a human being, in need thereof to treat symptoms associated with atrophic vaginitis. The invention describes both a safe and clinically effective formulation necessary to treat vaginal symptoms resulting from surgical menopause, iatrogenic menopause, natural menopause and conditions leading to Amenorrhea (uterus present) thus manifesting as menopause (See Table 3).

TABLE 3

| | |
|---|---|
| 1. | Anorexia Nervosa |
| 2. | Chromophobe Adenoma |
| 3. | Functional Hypothalamic Amenorrhea |
| 4. | Gonadal Failure |
| 5. | Gonadal Streaks |
| 6. | Gonadotrophin-Resistant Ovary Syndrome |
| 7. | Hypogonadotrophic Hypogonadism |
| 8. | Hypothalamic Dysfunction |
| 9. | Hypothalamic Failure |
| 10. | Isolated Gonadotrophin Deficiency |
| 11. | Pituitary Destruction |
| 12. | Polycystic Ovary Syndrome |
| 13. | Ovarian Destruction |
| 14. | Premature Ovarian Failure |
| 15. | Pure Gonadal Dysgenesis |
| 16. | Pituitary Failure |
| 17. | Hypothalamic etiology |
| 18. | Ovarian etiology |
| 19. | Pituitary etiology |
| 20. | Pituitary Dysfunction |

The pharmaceutical composition may be used to treat various conditions of the vagina, urethra and bladder including but not limited to pain, burning, irritation, itching, dryness, pressure, urinary frequency and incontinence. The compound, pharmaceutical composition, or unit dosage form of the present invention may be administered alone at appropriate dosages defined by routine testing in order to obtain greatest efficacy minimizing any potential adverse side effects.

In certain aspects of the present invention, the combination therapy may be used to treat bladder dysfunction, and more specifically overactive bladder. Lower urinary symptoms include dysuria, frequency, urgency, and incontinence (Simunic, et al. Int J Gynaecol Obstet 2003; 83: 187-197). Overactive bladder or hyperactive bladder, which is defined as bladder “urgency” or “frequency” with or without urge incontinence, usually with frequent nocturia.

Accordingly, the present invention may further include one or more anticholinergics, which inhibit transmission of parasympathetic nerve impulses and thereby reduce spasms of smooth muscle, for example, in the bladder. Anticholinergic compounds include but are not limited to muscarinic receptor antagonists, nicotinic receptor antagonists, and depolarizing neuromuscular blocking agents. Anticholinergics agents contemplated by the present invention include those known in the art, including for example but not limited to, darifenacin, dicyclomine, oxybutynin, and tolterodine. The anticholinergic agent may be used with estrogen, or with progesterone, or with the combination of estrogen and progesterone.

The daily dosage of the compound of the present invention may vary according to a variety of factors such as underlying disease states, the individual's condition, weight, age and the mode of administration. For vaginal administration, the pharmaceutical compositions can be provided in unit dosage forms containing most preferably from about 0.5 mg:25 mg per dose, preferably to about 1 mg:25 mg per dose, preferably 1 mg:30 mg per dose, preferably to about 1 mg:50 mg per dose, even up to about 1 mg:100 mg of the estrogen: progesterone of the present invention for the symptomatic adjustment of the dosage to the patient to be treated.

In contrast to other hormone replacement therapy protocols, vaginal administration may continue for at least 3 months, preferably at least 6 months, more preferably at least 12 months. In a specific embodiment, treatment will continue at least 18 months, more preferably at least 24 months. In a further embodiment, treatment is continuous for the lifetime of the patient. Specific formulations of estriol or micronized progesterone, and particularly both, are preferred for such long-term use.

### EXAMPLES

The following examples are merely illustrative of the present invention and they should not be considered as limiting the scope of the invention in any way.

### Example 1

### Estrogen/Progesterone Vaginal Suppository in Patients with Atrophic Vaginitis

The present example describes a Phase 1-2, open label, randomized, single blinded, placebo controlled, multiple dose trial of the safety profile of an estrogen/progesterone vaginal suppository (“JC-001”) in postmenopausal patients suffering from atrophic vaginitis.

The study objectives are as follows:

The objective of the trial is to assess, among postmenopausal women the efficacy between placebo, unopposed estrogen, and two combined estrogen-progesterone regimens for the treatment of atrophic vaginitis and assess their relative safety.

To compare the efficacy of the vaginal preparations with each other and with placebo in relieving the symptoms of atrophic vaginitis when efficacy will be measured by the improvement in vaginal atrophy measured by both objectively and subjectively. The objective measurement of improvement includes the measurement of vaginal pH and for the presence of vaginal Lactobacilli. The subjective measures of improvement will include the investigator's evaluation of the appearance of the vagina including vaginal mucosal pallor, petechiae, friability and dryness; and that of the patient assessment of symptoms relating to dryness and irritation.

To compare the safety of the vaginal preparations with each other and with placebo, in particular, effect of treatment on endometrial stimulation. The safety profile will include an assessment of endometrial stimulation measured by results of endometrial biopsy. The trial will report the endometrial histological findings in postmenopausal women who were randomized to receive placebo, unopposed estrogen and two combined estrogen-progesterone regimens.

The study population includes women of all races with a uterus and irrespective of prior hormone use are asked to participate in the study. Participants are between the ages of 45 and 64 at their randomization visit, and have ceased menstruation at least a year prior to entry. The participants have follicle-stimulating hormone (FSH) greater than or equal to 40 mIU/ml. Each participant will be informed of the possible side effects of the study design and the medical significance of these possible side effects. After this information is provided, signed consent is obtained from all participants.

The study is designed to randomize a total of 20 women, 5 in each of the study arms. Exclusion Criteria include the following:
1. The last menstrual period before the age of 44, or less than 12 months prior to randomization.
2. Serum FSH concentrations less than 40 mIU/ml.
3. A Body Mass Index greater than or equal to 40 kg/m2.
4. Use of the following drugs or agents: coumadin or heparin; menopausal hormones within 3 months of randomization; significant use of over-the-counter phytoestrogens within 3 months of randomization.
5. The patient doesn't have a diagnosis of atrophic vaginitis, as measured by a vaginal pH of less than 5. An investigator's evaluation of the vaginal appearance not consistent with a diagnosis of atrophic vaginitis (presence of normal mucosal color and normal rugosity). The participant's assessment of symptoms not relating to atrophy such as dryness or irritation.
6. A medical history of endometrial ablation.
7. A medical history of thromboembolic event associated with previous estrogen use.
8. Breast cancer or a mammogram that is positive or suspect for breast cancer at baseline or breast cancer occurring in an identical twin.
9. Endometrial cancer or endometrial hyperplasia based on clinical biopsy.
10. Myocardial infarction within 6 months of initial screening visit or coronary heart disease requiring antiarrhythmics or digitalis or congestive heart failure.
11. Stroke or TIA (ever).
12. Malignant melanoma (ever).
13. Any cancer (except nonmelanonomatous skin cancer) diagnosed less than 5 years prior to randomization.
14. Chronic liver disease.
15. Any other major life-threatening illness.
16. The patient is not able to demonstrate the ability to properly use the vaginal suppository prior to enrollment, doesn't understand English, is not able to cooperate with study procedures and is unlikely to remain with the study area for 1 year.

Estriol at a dose of 1 mg is given with the dosing schedule of maintenance 3 times per week after a loading dose and is given in suppository format. This dosing scheme is selected because (1) clinical data has shown that a lower dose of 0.5 mg has failed to restore the population of lactobacilli and has failed to reduce the vaginal pH in menopausal patients; (2) it is recommended as a dosing schedule to use a low dose or low potency estrogen given vaginally 3 times a week as maintenance after the loading dose; and (3) studies on estrogen tablets and vaginal rings provide insufficient data to recommend these alternatives for the treatment of atrophic vaginitis.

A specific progestational agent is also used, as it is known that the type of progesterone could markedly influence lipid levels. Micronized progesterone is selected, which is a naturally occurring progesterone rather than a synthetic progestin for safety reasons. Prior data has compared the bioavailability of orally and vaginally administered progesterone and shows that peak plasma progesterone concentrations for the two formulations are not significantly different and have a similar bioavailability. In addition, the data has shown that the relative potency for the ability to induce endometrial safety with the recommended progesterone dose for oral therapy to be 200 mg; and that with progesterone vaginal suspension to be 90 mg. Studies have demonstrated that at a dose of 100 mg micronized progesterone transvaginally 12 days/months resulted in a functional-like secretive endometrium.

Therefore, an approximate dose of 50 mg micronized progesterone and 25 mg micronized progesterone is used when evaluating the endometrial effects of vaginal hormone therapy in the current study. The treatment regimens selected for the study has four arms:
(1) Placebo;
(2) Estriol 1 mg;
(3) Estriol 1 mg and Micronized Progesterone 25 mg; and
(4) Estriol 1 mg and Micronized Progesterone 50 mg.

Patients randomized to the treatment group will receive JC-002 placebo as part of the single blinded.

Intravaginal placebo is composed of MBK Base-1.2500 gm. Intravaginal placebo is a suppository matching the JC-001 estriol/progesterone suppository. The identity of the test preparation is concealed on the masked portion of the label. Patients randomized to the placebo group will receive a suppository of JAB Base and self-administer intravaginal placebo. The drug formulations are as follows in Table 4:

TABLE 4

| Strength | 1 mg/25 mg | 1 mg/50 mg | 1 mg | Placebo |
|---|---|---|---|---|
| Estriol | 0.0010 gm/ml | 0.0010 gm/ml | 0.0010 gm/ml | 0 |
| Progesterone | 0.0250 gm/ml | 0.0500 gm/ml | 0 | 0 |
| Silica Gel | 0.0150 gm | 0.0150 gm | 0.0150 gm | 0 |
| JAB Base | 1.2431 gm | 1.2206 gm | 1.2656 gm | 1.2800 gm |
| Suppository | 1.2841 gm to | 1.2866 gm to | 1.2816 gm to | 1.2800 gm to |
| volume | volume | volume | volume | volume |
| Citric Acid 0.1%, | For pH | For pH | For pH | For pH |
| at 0.0013 gm | adjustment | adjustment | adjustment | adjustment |

Participants are randomized in equal numbers to one of the following treatments: vaginal suppository containing 1 mg estriol and 50 mg micronized progesterone per day for two weeks and then three times per week there after (n=5); vaginal suppository containing 1 mg estriol and 25 mg micronized progesterone per day for two weeks and the three times per week there after (n=5); vaginal suppository containing 1 mg estriol per day for two weeks and then three times per week there after (n=5); or placebo (n=5). The patients insert the suppository intravaginally once daily for 2 weeks. Thereafter, patients insert the suppository three times per week with at least a greater than 2-day interval between treatments to maintain therapeutic response.

Patients are evaluated for efficacy and safety at months 3, 6, and 12. Patients are also contacted by telephone at week 2 after the initial loading dose to assess any adverse events. At initial screening visit, a medical history is obtained and a general physical examination and pelvic examination is performed. Each participant completes a questionnaire regarding symptoms of urogenital atrophy. In addition, a vaginal pH will be measured with a pH meter and a vaginal culture will be obtained by rolling a swab across the lateral wall inside the vaginal introitus and promptly inoculated to isolate lactobacilli at baseline, 3, 6, and 12 months to assess efficacy. An endometrial biopsy will be performed at baseline, 3, 6, and 12 months to assess the safety profile (see further details under the section of endometrial histology procedures). Table 5 summarizes the collection of data.

TABLE 5
Data and Specimen Collection Schedule (0-12 months)

| | | Month | Month | Month |
|---|---|---|---|---|
| Parameter Assessed | Baseline | 3 | 6 | 12 |
| Gynecology and | X | | | |
| medical history | | | | |
| Complete physical | X | | | |
| examination | | | | |
| Vaginal pH | X | X | X | X |
| Vaginal lactobacilli | X | X | X | X |
| Vaginal atrophy | X | X | X | X |
| Vaginal dryness | X | X | X | X |
| Vaginal irritation | X | X | X | X |
| Endometrial biopsy | X | X | X | X |
| Adverse effects | | X | X | X |
| Performance evaluation | | X | X | X |
| Follicle stimulating | X | | | |
| hormone (FSH) | | | | |

Included among the data collection and procedures at annuals visits are a pelvic examination and cervical pap smear if needed. Unscheduled visits are conducted as required to respond to problems noted by participant or the investigator. Further, at each scheduled visit, a diary of symptoms, reports of vaginal bleeding, medication use, and interim illness are reviewed.

Endometrial tissue is obtained using standard biopsy techniques, without regard to the day of the women's menstrual cycle. The biopsies are performed with a Pipelle cannula. Biopsy results for women in whom the investigator is certain of entry into the uterus but is unable to obtain tissue (due to presumed atrophy) are classified as normal. Women in whom entry into the uterus is not possible (cervical stenosis or intolerance to the procedure) at baseline will not be assigned to a study arm. If this occurs at follow-up visits, the woman will discontinue study drug. Unscheduled biopsy is performed to evaluate abnormal or problematic vaginal bleeding, or as a follow-up to an earlier diagnosis of hyperplasia. Specimens will be fixed in 4% unbuffered formalin, and 4-um sections were stained with hematoxylin and eosin. The same pathologist, who is blinded to the patient's protocol regimen, will interpret the biopsy results. The criteria for the diagnosis of endometrial hyperplasia and the terminology used to classify endometrial hyperplasia will be used according to standard criteria.

Histology of endometrium collected at baseline, three months, six months and twelve months or unscheduled visits by biopsy, curettage, or hysterectomy.

### Example 2

### Formulation of Pharmaceutical Composition in Cream Form

The present example provides formulations of pharmaceutical compositions to treat symptoms associated with atrophic vaginitis as a vaginal cream. Table 6 summarizes the constituents and their amounts.

TABLE 6

| | | | | | | | Pla- |
|---|---|---|---|---|---|---|---|
| Strength | 1/25 mg/gm | | 1/50 mg/gm | | 1 mg/gm | | cebo |
| Estriol | 0.0010 | gm | 0.0010 | gm | 0.0010 | gm | 0 |
| Progesterone | 0.0250 | gm | 0.0500 | gm | 0 | | 0 |
| Propylene Glycol | 0.0250 | ml | 0.0500 | ml | 0.005 | ml | 0 |
| (wetting agent) | | | | | | | |
| JC Base | 0.949 | gm | 0.899 | gm | 0.994 | gm | 0 gm |
| (Base B and Base M) | | | | | | | |
| Base B is emollient | | | | | | | |
| cream Base M is | | | | | | | |
| Vitamin E Acetate | | | | | | | |
| USP Liquid (1 IU/mg) | | | | | | | |

The total volume of each dose is 1 gm for every strength.

### Example 3

### Formulation of Pharmaceutical Composition in Cream Form

The present example provides formulations of a pharmaceutical composition to treat symptoms associated with atrophic vaginitis as a vaginal cream. Table 7 summarizes the constituents and their amounts.

TABLE 7

| | | |
|---|---|---|
| Strength | 1/25 mg/gm | 1/50 mg/gm |

| | | | | |
|---|---|---|---|---|
| Estriol | 0.0010 | gm | 0.0010 | gm |
| Progesterone | 0.0250 | gm | 0.0500 | gm |
| Propylene Glycol | 0.0250 | ml | 0.0500 | ml |
| (wetting agent) | | | | |
| JC Base | 0.949 | gm | 0.899 | gm |
| (Base B and Base M) | | | | |
| Base B is PCCA's Versabase | | | | |
| Base M is Vitamin E Acetate | | | | |
| USP Liquid (1 IU/mg) | | | | |

### Example 4

### Formulation of Pharmaceutical Composition in Cream Form

The present example provides formulations of a pharmaceutical composition to treat symptoms associated with atrophic vaginitis as a vaginal suppository. Table 8 summarizes the constituents and their amounts.

TABLE 8

| Strength | 1 mg/25 mg | | 1 mg/50 mg | |
|---|---|---|---|---|
| Estriol | 0.0010 | gm/ml | 0.0010 | gm/ml |
| Progesterone | 0.0250 | gm/ml | 0.0500 | gm/ml |
| Silica Gel | 0.0150 | gm | 0.0150 | gm |
| JAB Base | 1.2431 | gm | 1.2206 | gm |
| Suppository | 1.2841 | gm to volume | 1.2866 | gm to volume |
| volume | | | | |

| | | |
|---|---|---|
| Citric Acid 0.1%, | For pH adjustment | For pH adjustment |
| at 0.0013 gm | | |

### Example 5

### Efficacy and Safety Study with Vaginal Estriol and Progesterone in a Single Dosage Unit for the Treatment of Atrophic Vaginitis in Menopausal Patient

The formulation of the combination of estriol and progesterone by compounding estriol and progesterone and administering it as a single dosage unit to eleven (11) patients was explored. Patients ranged in age from (51 years) to (75 years), with a mean age of (59 years). All women presented with vaginal atrophy symptom vaginal dryness. All women were treated using a combination estriol and progesterone vaginal suppository to be given once per day for two weeks followed by a maintenance regimen of two times per week. Five women were given the dosage of estriol 1 mg and progesterone 25 mg. Six women were given the dosage of estriol 1 mg and progesterone 30 mg. Blood samples were collected approximately 3 to 5 hours after insertion of the suppository.

As shown in Table 9, patients in the study reported improvement in the vaginal atrophy symptom of vaginal dryness after treatment with the combination estriol and progesterone suppository by month 3 of treatment. Both estriol 1 mg/progesterone 25 mg (n=5) and estriol 1 mg/progesterone 30 mg (n=6) treatments resulted in an improvement in the vaginal dryness index (rating scale) when compared to the baseline values (where “0” means no dryness and “10” means extreme dryness). The gynecologic evaluation also included a vaginal pH assessment. Vaginal pH was measured using an indicator strip. There was no significant difference between the 2 dose groups in median pH and vaginal dryness values at baseline or at the 3-month follow-up, or between the changes in these values (Table 9). There was a significant difference in the median change between baseline and 3 months in pH and vaginal dryness values within each dosage group (Table 9).

TABLE 9
Clinical modifications induced by intravaginal Estriol/Progesterone
therapy: Vaginal pH and Vaginal Dryness

| | Estriol 1/ | | Estriol 1/ | | |
|---|---|---|---|---|---|
| | Progesterone 25 | | Progesterone 30 | | |
| | Mean + sd | Median | Mean + sd | Median | P* |
| pH Baseline | 7.2 + 0.6 | 7.5 | 6.8 + 0.6 | 6.5 | 0.3 |
| pH 3 months | 4.9 + 0.6 | 4.6 | 4.8 + 0.2 | 4.75 | 0.5 |
| pH Change | 2.3 + 0.7 | 2.4 | 2.0 + 0.7 | 1.75 | 0.4 |
| | | P^{†} = 0.03 | | P^{†} = 0.02 | |
| Vaginal Dryness | 8.2 + 0.8 | 8.0 | 8.0 + 0.8 | 8.0 | 0.7 |
| Baseline | | | | | |
| Vaginal Dryness | 1.8 + 2.1 | 1.0 | 1.9 + 0.7 | 2.0 | 0.4 |
| 3 months | | | | | |
| Vaginal Dryness | 6.3 + 1.6 | 7.0 | 6.1 + 0.9 | 6.0 | 0.2 |
| Change | | | | | |
| | | P^{†} = 0.02 | | P^{†} = 0.01 | |

*P-value from Mann-Whitney test for difference in medians between the two doses
^{†}P-value from Wilcoxon Signed rank test for change in medians within each dose

There was some absorption of progesterone through the vaginal mucosa as demonstrated by evidence of serum progesterone levels, although levels did not vary greatly and fell well within normal range (normal luteal phase levels range vary from 1.8 ng/ml to 26 ng/ml). These data indicate systemic bioavailability for progesterone that appears to yield levels closely confined to luteal phase progesterone levels. This data would be consistent with the doses necessary as reported in the medical literature sufficient to have an anti-proliferative effect reported to occur with an estrogen stimulated postmenopausal endometrium. Table 10 summarizes the progesterone serum concentrations.

TABLE 10

| Patient | Progesterone dose | Serum (ng/ml) |
|---|---|---|
| 1 | 25 mg | 4.8 |
| 2 | 25 mg | 8.8 |
| 3 | 25 mg | 4.2 |
| 4 | 25 mg | 5.4 |
| 5 | 25 mg | 5.7 |
| 6 | 30 mg | 6.3 |
| 7 | 30 mg | 2.0 |
| 8 | 30 mg | 5.6 |
| 9 | 30 mg | 2.9 |
| 10 | 30 mg | 5.6 |
| 11 | 30 mg | 5.2 |

Estrogen-deficient women received treatment regimens (estriol 1 mg/progesterone 25 mg [n=5]; estriol 1 mg/progesterone 30 mg [n=5]) twice per week for approximately twelve months and a mammogram was obtained after one year of treatment. All ten mammogram results were normal. The results indicate that there is no increase risk of breast cancer with the combination vaginal hormone replacement therapy, which is in contrast to oral or transdermal combination hormone replacement therapy. Table 11 summarizes the mammogram findings.

TABLE 11

| | Estriol 1 mg/ | Estriol 1 mg/ |
|---|---|---|
| Result | Progesterone 25 mg | Progesterone 30 mg |
| Normal | 5 | 5 |
| Increased breast tissue density | 0 | 0 |
| Abnormal | 0 | 0 |
| Total | 5 | 5 |

### Example 6

### Efficacy and Safety Study with Vaginal Estriol and Progesterone in a Single Dosage Unit for the Treatment of Atrophic Vaginitis in Menopausal Patients

A pilot study was conducted to investigate whether the combination of an estriol and progesterone vaginal suppository is effective and safe in the treatment of atrophic vaginitis in postmenopausal women.

The test drug formulation is located in Table 12. Participants were given the following treatment: Vaginal suppository containing 1 mg estriol and 30 mg progesterone per day for two weeks and then three times per week thereafter (n=19). The collection of data is summarized in Table 13.

TABLE 12
Test Drug Formulation

| | 1 mg/30 mg |
|---|---|
| Hormone Strength | (JC-001) |
| Micronized Estriol | 0.0010 gm |
| Micronized Progesterone | 0.0300 gm |
| Silica Gel | 0.0150 gm |
| Base JAB: (fatty base) | 1.2386 gm |
| Suppository Volume | 1.2846 gm |
| Citric Acid 0.1% at | For pH Adjustment |
| 0.0013 gm | |

TABLE 13
Data Collection Schedule (0-6 months)

| | | Week | Week | Week |
|---|---|---|---|---|
| Variable Assessed | Baseline | 2 | 12 | 24 |
| Medical History | X | | | |
| Vaginal pH | X | | X | X |
| Urinalysis | X | | X | X |
| Vaginal cytology | X | | X | X |
| Self-assessment of | X | | X | X |
| urinary frequency | | | | |
| Self-assessment of | X | | X | X |
| libido | | | | |
| Self-assessment of | X | | X | X |
| vaginal dryness | | | | |
| Serum Estriol and | X | X | X | X |
| Progesterone | | | | |
| Endometrial biopsy | X | | | X |
| Serum Follicle | X | | | |
| Stimulating Hormone | | | | |
| Physical Exam | X | | X | X |

This study enrolled a sample of 19 participants. All 19 subjects had symptoms of atrophic vaginitis. Vaginal and endometrial atrophy were present in all cases.

A previous study of postmenopausal women with atrophic vaginitis reported mean pre-treatment Vaginal Maturation Index (VMI) and pH values of 39.5 and 6.2, respectively (Marx et al., Maturitas 2004; 47:47-54). Based on these data, assuming that the standard deviations of the differences are no greater than 14 for VMI and 0.8 for vaginal pH, a sample size of 18 will have greater than 80% power to detect a 25% change in the VMI and a 10% change in vaginal pH. In addition, if the true rate of endometrial hyperplasia is 1%, a sample size of 18 women will have 98.6% power to exclude rates greater than 25% (i.e. the probability of observing only 0 or I event is less than 0.05 when the true rate is 25%, while the probability is 0.986 when the true rate is 1%).

The primary endpoints in this study included changes in the Vaginal Maturation Index, self-assessment of vaginal dryness, urinary frequency and libido and vaginal pH defined as the difference between the baseline and the 3- and 6-month follow-up measurements. The secondary endpoints included the presence of an abnormal endometrial biopsy result at 6 months, defined as histological evidence of prolonged estrogenic effect or endometrial hyperplasia, and changes in serum estriol and progesterone concentrations defined as the difference between the baseline and the 2-week, the 3-month and 6-month follow-up measurements.

Descriptive statistics provided for the continuous study endpoints included mean, median, standard deviation, and 95% confidence intervals. Descriptive statistics provided for categorical endpoints included frequencies, percents, and 95% confidence intervals. Missing values of a variable were imputed using the last observed value for the participant. Descriptive statistics were provided with and without imputation of missing values. Zero cases of endometrial hyperplasia in the hormone regimens in this study were interpreted as a long run risk that is no greater than 14% at the 95% confidence level based on the equation (1−Maximum Risk)ⁿ=0.05 (Hanely et al., JAMA 1983, 249:1743-45).

Though, this number is not reflective of the long run risk because an estrogen and progesterone vaginal product has not been studied long term. The long run risk seen with combination estrogen and progesterone oral therapy had rates of endometrial hyperplasia that were less 1% over a 3-year study. The anticipated results would include similar rates of endometrial hyperplasia (less then 1%) when using a combination estrogen and progesterone vaginal product.

No adverse effects occurred during the 3-month treatment period. All subjects returned for evaluation after 3 months of treatment, and 18 reported satisfactory relief of vaginal dryness symptom. One subject reported mild subjective relief of vaginal dryness symptom, despite objective improvement in vaginal atrophy. The treatment resulted in a significant improvement in the vaginal dryness index between the enrollment and the 12-week visit. There was a significant improvement in vaginal maturation index was seen between the enrollment and the 12-week visit. There was a significant improvement in the pH change between enrollment and the 12-week visit. There was a significant improvement in overactive bladder symptom urinary frequency between enrollment and 12-week visit. In addition, there was a significant improvement in hypoactive desire phase disorder (libido) between enrollment and 12-week visit. Tables 14 and 15 summarize the clinical modifications with an estriol and progesterone vaginal combination hormone therapy.

TABLE 14
Median symptom scores, estriol and progesterone levels, and paired
differences between the enrollment and 2- and/or 12-week visits.

| | | Me- | | |
|---|---|---|---|---|
| | N | dian* | Range* | P^{†} |
| VMI Enrollment | 19 | 40.0 | 0-55.0 | |
| VMI 12-week | 19 | 57.5 | 47.5-75.0 | |
| Paired Difference | 19 | 25.0 | 0-50.0 | <0.001 |
| pH Enrollment | 19 | 6.0 | 5.0-7.5 | |
| pH 12-week | 19 | 4.5 | 4.2-5.3 | |
| Paired Difference | 19 | −1.5 | −2.5-−0.3 | <0.001 |
| Vaginal Dryness Enrollment | 19 | 9.0 | 6.0-10.0 | |
| Vaginal Dryness 12-week | 19 | 2.0 | 0-7.0 | |
| Paired Difference | 19 | −5.0 | −9.0-−2.0 | <0.001 |
| Libido Enrollment | 11 | 4 | 1-6.0 | |
| Libido 12-week | 11 | 0 | 0-5.0 | |
| Paired Difference | 11 | −2.0 | −5.0-−1.0 | 0.003 |
| Urinary Frequency Enrollment | 12 | 3.5 | 1-5.0 | |
| Urinary Frequency 12-week | 12 | 0 | 0-1.0 | |
| Paired Difference | 12 | −3.5 | −4.0-−1.0 | 0.002 |
| Serum Estriol Enrollment (ng/ml) | 19 | 0.1 | 0.1-0.1 | |
| Serum Estriol 2-week Pre-insertion | 6 | 0.1 | 0.1-0.16 | |
| (ng/ml) | | | | |
| Paired Difference (Pre-insertion - | 6 | 0 | 0-0.06 | 0.2 |
| Enrollment) | | | | |
| Serum Estriol 2-week Post-insertion | 6 | 0.16 | 0.1-0.35 | |
| (ng/ml) | | | | |
| Paired Difference (Post-insertion - | 6 | 0.06 | 0-0.25 | 0.04 |
| Enrollment) | | | | |
| Serum Estriol Enrollment (ng/ml) | 19 | 0.1 | 0.1-0.1 | |
| Serum Estriol 12-week Pre-insertion | 13 | 0.1 | 0.1-0.25 | |
| (ng/ml) | | | | |
| Paired Difference (Pre-insertion - | 13 | 0 | 0-0.15 | 0.1 |
| Enrollment) | | | | |
| Serum Estriol 12-week Post-insertion | 13 | 0.25 | 0.1-0.71 | |
| (ng/ml) | | | | |
| Paired Difference (Post-insertion - | 13 | 0.15 | 0-0.61 | 0.003 |
| Enrollment) | | | | |
| Serum Progesterone Enrollment | 19 | 0.5 | 0.3-1.2 | |
| (ng/ml) | | | | |
| Serum Progesterone 2-week Pre- | 6 | 4.2 | 0.9-8.3 | |
| insertion (ng/ml) | | | | |
| Paired Difference (Pre-insertion - | 6 | 3.6 | 0.2-7.8 | 0.03 |
| Enrollment) | | | | |
| Serum Progesterone 2-week Post- | 6 | 6.6 | 4.2-10.0 | |
| insertion (ng/ml) | | | | |
| Paired Difference (Post-insertion - | 6 | 6.0 | 3.5-9.5 | 0.03 |
| Enrollment) | | | | |
| Serum Progesterone Enrollment | 19 | 0.5 | 0.3-1.2 | |
| (ng/ml) | | | | |
| Serum Progesterone 12-week Pre- | 13 | 1.2 | 0.8-9.0 | |
| insertion (ng/ml) | | | | |
| Paired Difference (Pre-insertion - | 13 | 0.8 | −0.3-8.6 | 0.004 |
| Enrollment) | | | | |
| Serum Progesterone 12-week Post- | 15 | 7.9 | 3.7-15.3 | |
| insertion (ng/ml) | | | | |
| Paired Difference (Post-insertion - | 15 | 6.9 | 3.0-14.9 | 0.001 |
| Enrollment) | | | | |

*A negative value indicates a decrease from enrollment whereas a positive value indicates an increase from enrollment
^{†}P-value from Wilcoxon signed rank test which compared the enrollment value to the 2- and/or 12-week value for each participant

TABLE 15
Presence of libido and urinary frequency symptoms
at enrollment and the 12-week visit.

12-week

| Symptom | Present (n) | Absent (n) | P* |
|---|---|---|---|
| Libido Enrollment | | | 0.02 |
| Present | 4 | 7 | |
| Absent | 0 | 8 | |
| Urinary Frequency Enrollment | | | 0.001 |
| Present | 1 | 11 | |
| Absent | 0 | 7 | |

*P-value from McNemar's test which compared the presence and absence of symptom between the enrollment and 12-week visit for each participant

In addition, estrogen-deficient women who received treatment regimen estriol 1 mg/progesterone 30 mg three times per week for approximately twelve weeks (3 months) had a blood sample obtained approximately 4 to 5 hours after insertion of the suppository. There was some absorption of progesterone through the vaginal mucosa as demonstrated by evidence of serum progesterone levels, although levels did not vary greatly and fell well within normal range (normal luteal phase levels range vary from 1.8 ng/ml to 26 ng/ml). These data indicate systemic bioavailability for progesterone that appears to yield levels closely confined to luteal phase progesterone levels. Again, this data (mean serum concentration of 7.7 ng/ml) would be consistent with the doses necessary as reported in the medical literature sufficient to have an anti-proliferative effect (greater than 5 ng/ml) reported to occur with an estrogen stimulated postmenopausal endometrium. Table 16 summarizes the serum progesterone concentrations following administration of a combination vaginal hormone therapy given three times per week. Tables 17 and 18 demonstrate that there were no significant differences between the enrollment progesterone concentration and the pre-insertion concentration at week 2 and at week 12, therefore suggesting minimal systemic absorption. Overall, these results indicate that the systemic effects of progesterone administration would be substantially less than that of a dose given orally.

TABLE 16
Serum Progesterone Concentrations following Administration
of an Estriol/Progesterone Vaginal Suppository three
times per week to Postmenopausal Women.

| Patient | Progesterone dose | Serum (ng/ml) |
|---|---|---|
| 1 | 30 | 8.1 |
| 2 | 30 | 5.2 |
| 3 | 30 | 7.9 |
| 4 | 30 | 4.1 |
| 5 | 30 | 8.1 |
| 6 | 30 | 15.3 |
| 7 | 30 | 8.4 |
| 8 | 30 | 10.6 |
| 9 | 30 | 5.3 |
| 10 | 30 | 8.2 |
| 11 | 30 | 5.7 |
| 12 | 30 | 10.7 |
| 13 | 30 | 6.8 |
| 14 | 30 | 7.6 |
| 15 | 30 | 3.7 |
| Mean | | 7.7 |

TABLE 17
Presence and absence of progesterone level >5 ng/ml between
the enrollment and 2-week visits for each participant

| | Enrollment | | |
|---|---|---|---|
| | >5 ng/ml | <5 ng/ml | |
| Symptom | (n) | (n) | P* |
| Progesterone 2-week Pre-insertion | | | 1.0 |
| ≧5 ng/ml | 0 | 1 | |
| <5 ng/ml | 0 | 5 | |
| Progesterone 2-week Post-insertion | | | 0.06 |
| ≧5 ng/ml | 0 | 5 | |
| <5 ng/ml | 0 | 1 | |

*P-value from McNemar's test which compared the presence and absence of progesterone level ≧5 ng/ml between the enrollment and 2-week visits for each participant

TABLE 18
Presence and absence of progesterone level >5 ng/ml between
the enrollment and 12-week visits for each participant

| | Enrollment | | |
|---|---|---|---|
| | ≧5 ng/ml | <5 ng/ml | |
| Symptom | (n) | (n) | P* |
| Progesterone 12-week Pre-insertion | | | 0.1 |
| ≧5 ng/ml | 0 | 4 | |
| <5 ng/ml | 0 | 9 | |
| Progesterone 12-week Post-insertion | | | <0.001 |
| ≧5 ng/ml | 0 | 13 | |
| <5 ng/ml | 0 | 2 | |

*P-value from McNemar's test which compared the presence and absence of progesterone level ≧5 ng/ml between the enrollment and 12-week visits for each participant

Five patients underwent an endometrial biopsy (EMB) after 6 months of treatment. The results were consistent an anti-proliferative effect on the uterus, which is consistent with that reported with a combination oral or transdermal hormone replacement therapy. Therefore, the vaginal dose of 30 mg progesterone was sufficient to have an anti-proliferative effect on estrogen stimulated postmenopausal endometrium. Table 19 summarizes the endometrial changes.

TABLE 19
Summary of Endometrial Biopsy Changes Since Normal Baseline
to Most Extreme Abnormal Results at Month 6.

| Results | Estriol 1 mg/Progesterone 30 mg |
|---|---|
| Normal | 5 |
| Simple (cystic) hyperplasia | 0 |
| Complex (adenomatous) hyperplasia | 0 |
| Atypia | 0 |
| Adenocarcinoma | 0 |
| Total | 5 |

Measurement of estriol levels with the administration of an oral route has shown significantly greater systemic level of the hormone with oral administration. Table 20 demonstrates that the dose of 1 mg estriol that converted vaginal cytology and vaginal pH to premenopausal values showed no significant differences between serum concentration at week-2 and again at week-12 at pre-insertion. Overall, these results indicate that the systemic effects of estriol administration would be substantially less than that of a dose given orally.

TABLE 20
Median estriol levels and paired differences between
the enrollment and 2- and 12-week visits.

| | | Me- | | |
|---|---|---|---|---|
| | N | dian* | Range | P^{†} |
| Serum Estriol Enrollment (ng/ml) | 19 | 0.1 | 0.1-0.1 | |
| Serum Estriol 2-week Pre-insertion | 6 | 0.1 | 0.1-0.16 | |
| (ng/ml) | | | | |
| Paired Difference (Pre-insertion - | 6 | 0 | 0-0.06 | 0.2 |
| Enrollment) | | | | |
| Serum Estriol 2-week Post-insertion | 6 | 0.16 | 0.1-0.35 | |
| (ng/ml) | | | | |
| Paired Difference (Post-insertion - | 6 | 0.06 | 0-0.25 | 0.04 |
| Enrollment) | | | | |
| Serum Estriol Enrollment (ng/ml) | 19 | 0.1 | 0.1-0.1 | |
| Serum Estriol 12-week Pre-insertion | 13 | 0.1 | 0.1-0.25 | |
| (ng/ml) | | | | |
| Paired Difference (Pre-insertion - | 13 | 0 | 0-0.15 | 0.1 |
| Enrollment) | | | | |
| Serum Estriol 12-week Post-insertion | 13 | 0.25 | 0.1-0.71 | |
| (ng/ml) | | | | |
| Paired Difference (Post-insertion - | 13 | 0.15 | 0-0.61 | 0.003 |
| Enrollment) | | | | |

^{†}P-value from Wilcoxon signed rank test which compared the enrollment value to the 12-week value for each participant

In summary, the data showed an improvement between mean baseline and month 3 in vaginal maturation index (n=19); pH (n=19); vaginal dryness rating (n=19); libido (n=11); and urinary frequency (n=12). Six month EMB on 5 patients demonstrated an antiproliferative effect. 15 patients with serum progesterone levels indicated an antiproliferative effect. 13 patients with estriol serum levels showed minimal systemic absorption. Of note, 10 patients on the test drug for a year had no change in mammogram findings.

The present invention is not to be limited in scope by the specific embodiments described herein. Indeed, various modifications of the invention in addition to those described herein will become apparent to those skilled in the art from the foregoing description and the accompanying figures. Such modifications are intended to fall within the scope of the appended claims.

It is further to be understood that all values are approximate, and are provided for description.

Patents, patent applications, publications, product descriptions, and protocols are cited throughout this application, the disclosures of which are incorporated herein by reference in their entireties for all purposes.

1. A pharmaceutical composition for vaginal administration to a subject in need thereof comprising a therapeutically effective amount of an estrogen compound, a therapeutically effective amount of a progesterone compound, and a therapeutically effective amount of a pharmaceutically acceptable carrier for vaginal administration, wherein the composition is useful in treatment of urogenital symptoms associated with atrophic vaginitis.

2. The pharmaceutical composition according to claim 1, wherein the composition is prepared as a vaginal suppository.

3. The pharmaceutical composition according to claim 1, wherein the estrogen compound is micronized estriol.

4. The pharmaceutical composition according to claim 1, wherein the progesterone compound is micronized progesterone.

5. The pharmaceutical composition according to claim 1, wherein the estrogen compound is micronized estriol and wherein the progesterone compound is micronized progesterone.

6. The pharmaceutical composition according to claim 3, wherein the micronized estriol is present in an amount of about 1 mg per dose.

7. The pharmaceutical composition according to claim 3, wherein the micronized estriol is present in amounts from about 0.01 mg to about 10 mg, per dose.

8. The pharmaceutical composition according to claim 7, wherein the micronized estriol is present in amounts from about 0.25 mg to about 1.0 mg, per dose.

9. The pharmaceutical composition according to claim 4, wherein the micronized progesterone is present in amounts from about 5 mg to 500 mg per dose.

10. The pharmaceutical composition according to claim 9, wherein the micronized progesterone is present in amounts from about 25 mg to 50 mg per dose.

11. The pharmaceutical composition according to claim 5, wherein the micronized estriol and micronized progesterone are present in amounts of about 1 mg:25 mg respectively per dose.

12. The pharmaceutical composition according to claim 5, wherein the micronized estriol and micronized progesterone are present in amounts of about 1 mg:30 mg respectively per dose.

13. The pharmaceutical composition according to claim 5, wherein the micronized estriol and micronized progesterone are present in amounts of about 1 mg:50 mg respectively per dose.

14. The pharmaceutical composition according to claim 1, further comprising at least one constituent selected from the group consisting of additives, pharmaceutically acceptable carriers, fatty acid base, a preservative, a dye, a binder, a suspending agent, a dispersing agent, a colorant, a disintegrant, an excipient, a diluent, a lubricant, a plasticizer, oils, and mixtures thereof.

15. The pharmaceutical composition according to claim 4, wherein the micronized progesterone is given in a therapeutically effective dose to reduce concomitant liability of adverse uterine effects associated with long-term unopposed estrogen administration during menopause.

16. The pharmaceutical composition according to claim 1, wherein the composition further comprises a suspending agent.

17. The pharmaceutical composition according to claim 16, wherein the suspending agent is micronized silica gel.

18. The pharmaceutical composition according to claim 17, wherein the amount of micronized silica gel is 0.020 gm per unit dose.

19. The pharmaceutical composition of claim 1, wherein the composition further comprises a fatty acid base.

20. The pharmaceutical composition of claim 19, wherein the fatty acid base is composed of JAB base per suppository.

21. A method of treating urogenital symptoms of atrophic vaginitis, which comprises vaginally administering a pharmaceutical composition comprising therapeutically effective amounts of an estrogen compound and a progesterone compound.

22. The method according to claim 21, wherein the estrogen is a micronized estriol.

23. The method according to claim 21, wherein the progesterone is micronized progesterone.

24. The method according to claim 21, wherein the estrogen is a micronized estriol and wherein the progesterone is micronized progesterone.

25. The method according to claim 23, wherein the therapeutically effective amount of the progesterone is effective to reduce concomitant liability of adverse uterine effects associated with long-term unopposed estrogen administration during menopause.

26. The method according to claim 21, wherein the incidence of side effects associated with antimuscarinic treatment is reduced.

27. The method according to claim 24, wherein the amount of 0.5 mg micronized estriol combined with 25 mg micronized progesterone given vaginally causes an antiproliferative effect on an endometrium.

28. The method according to claim 24, wherein the estrogen and progesterone are present in a dose amounts of 1 mg micronized estriol:50 mg micronized progesterone, wherein vaginal administration causes an antiproliferative effect on an endometrium.

29. The method according to claim 24, wherein the amount of 1 mg micronized estriol combined with 25 mg micronized progesterone given vaginally causes an antiproliferative effect on an endometrium.

30. The method according to claim 24, wherein the estrogen and progesterone are present in a dose amounts of 1 mg micronized estriol:30 mg micronized progesterone, wherein vaginal administration causes an antiproliferative effect on an endometrium.

31. The method according to claim 21, wherein administration is continued for at least 3 months.

32. The method according to claim 31, wherein administration is continued for at least 6 months.

33. The method according to claim 32, wherein administration is continued for at least 12 months.

34. The method according to claim 33, wherein administration is continued for at least 18 months.

35. The method according to claim 34, wherein administration is continued for at least 24 months.

36. The method according to claim 24, wherein the estrogen and progesterone are present in dose amounts of 1.0 mg micronized estriol: 100 mg micronized progesterone wherein vaginal administration induces a full secretory endometrium resulting in withdrawal bleeding.

37. The method according to claim 24, wherein the estrogen and progesterone are present in dose amounts of 1 mg micronized estriol:50 mg micronized progesterone wherein vaginal administration leaves the endometrium partially secretory resulting in very light irregular bleeding and no withdrawal bleeding.

38. The method according to claim 24, wherein the estrogen and progesterone are present in dose amounts of 1 mg micronized estriol:30 mg micronized progesterone wherein vaginal administration leaves the endometrium partially secretory resulting in very light irregular bleeding and no withdrawal bleeding.

39. The method according to claim 24, wherein the estrogen and progesterone are present in dose amounts of 1 mg micronized estriol:25 mg micronized progesterone wherein vaginal administration leaves the endometrium partially secretory resulting in no irregular bleeding and no withdrawal bleeding.

40. The method of claim 21, wherein the estrogen compound and progesterone compound are administered as a vaginal suppository or vaginal cream.

41. The method according to claim 21, wherein the pharmaceutical compositions in administered in therapeutically effective amounts to reduce symptoms of overactive bladder.

42. The method of claim 41, wherein the symptoms of overactive bladder include frequency, urgency, nocturia, and urge incontinence.

43. The pharmaceutical composition of claim 1 further comprising an anticholinergic agent.

44. The method of claim 21, wherein the pharmaceutical composition further comprises an anticholinergic agent.

45. A suppository for vaginal administration, the suppository comprising:
a) about 0.01 mg to about 10 mg estriol;
b) about 5 mg to about 500 mg progesterone;
c) about 1.0 g to about 2.0 g of a fatty acid base; and
d) about 0.01 g to about 0.02 g silica gel.

46. The suppository of claim 45, wherein the fatty acid base comprises about 0.5 g to about 1.0 g PEG-8 distearate and about 0.5 g to about 1.0 g hydrogenated vegetable oil.

47. The suppository of claim 45 further comprising at least one of a preservative, a dye, a binder, a dispersing agent, a colorant, a disintegrant, an excipient, a diluent, a lubricant, a plasticizer, or an oil.

48. The suppository of claim 45, wherein the suppository comprises about 1 mg estriol and about 25 mg to about 50 mg progesterone.

49. A cream for vaginal administration, the cream comprising:
a) about 0.01 mg to about 10 mg estriol;
b) about 5 mg to about 500 mg progesterone;
c) about 0.5 g to about 1.0 g emollient cream;
d) about 0.025 mL to about 0.05 mL propylene glycol; and
e) about 0.01 g to about 0.02 g silica gel.

50. The cream of claim 49 further comprising at least one of a preservative, a dye, a binder, a dispersing agent, a colorant, a disintegrant, an excipient, a diluent, a lubricant, a plasticizer, or an oil.

51. The cream of claim 49, wherein the cream comprises about 1 mg estriol and about 25 mg to about 50 mg progesterone.

52. A method of treating menopause symptoms, the method comprising vaginally administering the suppository of claim 45.

53. A method of treating menopause symptoms, the method comprising vaginally administering an effective amount of the cream of claim 49.
